(19) United States
(12) Plant Patent Application Publication
(10) Pub. No.: US 2008/0141406 P1
(43) Pub. Date: Jun. 12, 2008

(54) Loblolly pine tree named 'CF Q7766'

(76) Inventors: John Pait, Atlanta, GA (US); Plamen Denchev, Victoria (CA); Stephen Attree, Victoria (CA); Robert J. Weir, Cary, NC (US); Andy Benowicz, Victoria (CA)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/635,199

(22) Filed: Dec. 7, 2006

(51) Int. Cl. A01H 7/00

(52) U.S. Cl. PLT/213

(57) ABSTRACT

A new and distinctive variety of a loblolly pine tree which has been denominated varietally as ‘CF Q7766’ which is distinguished by great resistance to fusiform rust and pitch canker, exceptionally high growth rate, excellent stem straightness, distinctive very long internodes, narrow crown.

### LATIN NAME

Pinus taeda

### VARIETY DENOMINATION

CF Q7766

### BACKGROUND

A new variety of loblolly pine tree (Pinus taeda), has been discovered. This selection has been designated as CF Q7766.

This new variety is a progeny of a second generation selection pollinated by a first generation selection. Female parent is an open pollinated progeny of Georgetown County, South Carolina first generation selection. Male parent is a first generation selection made in Barnwell County, South Carolina.

Cross pollination occurred in early 1998 followed by induction and cryopreservation of embryogenic tissue in 1999. First somatic seedlings were produced in 2000 and planted in early 2001 in seven field experiments. A total of 47 ramets were planted ranging from 4 to 8 ramets per field experiment. The field experiments are located in Mississippi, Florida, Georgia and South Carolina.

### BRIEF SUMMARY

A new and distinct cultivar of loblolly pine (Pinus taeda) is distinctly characterized by great resistance to fusiform rust and pitch canker, exceptionally high growth rate, excellent stem straightness, distinctive very long internodes, narrow crown, and which is mature for commercial harvesting sooner than conventionally grown trees under the ecological conditions prevailing in the Piedmont, Atlantic and Gulf Coastal Plains, and Mid-Continent regions of the United States.

The Pinus taeda plants of this variety were asexually propagated using an advanced form of micropopagation called somatic embryogenesis carried out at CellFor's production facility in Victoria, Canada. Somatic embryogenesis uses a complex process which relies on the splitting of one embryo into many identical embryos. Somatic embryos can then be grown into plants which are all identical genetically. The asexual propagation occurs at an earlier stage in the plant's life cycle than most other micropropagated plants. The detailed methods for somatic embryogenesis used for asexually propagating conifers in general are described in U.S. Pat. No. 6,372,496 and for loblolly pine in particular in US Patent Application 2004/0203150.

### BRIEF DESCRIPTION OF THE DRAWINGS

The drawings are color photographs showing the new variety of loblolly pine including the long internode length.

FIG. 1 is a photograph showing CF Q7766 ramet # 7 planted in Holt, Ga. The picture was taken after five field growing seasons. The picture shows distinctive long stem sections of clearwood between the whorls (long internodes), small to medium branch size (relative to the stem size), flat to medium angle between the branches and the stem and great stem straightness.

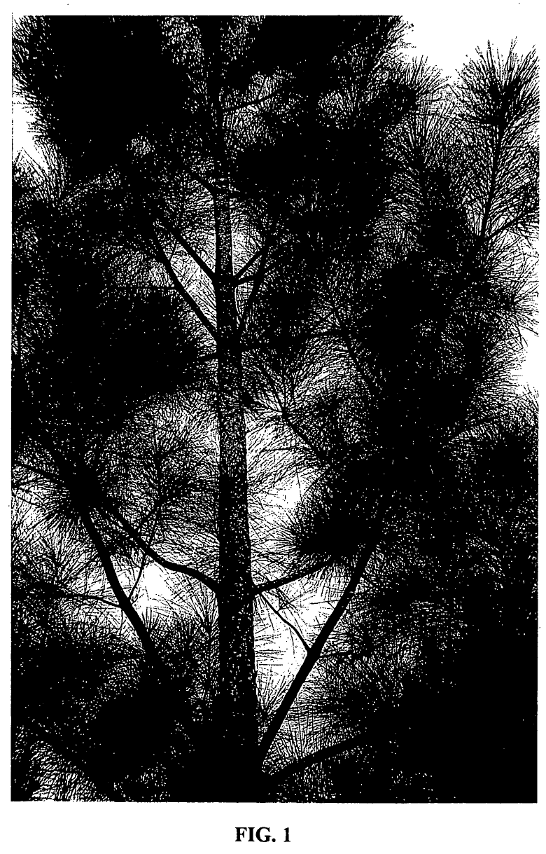

FIG. 2 is a photograph showing CF Q7766 ramet # 4 planted in Winokur, Ga. The picture was taken after five field growing seasons. The picture shows superiority of growth, narrow tree crown relative to the size of this tree and the size of surrounding neighbors, and distinctive long internodes seen particularly well in the upper crown.

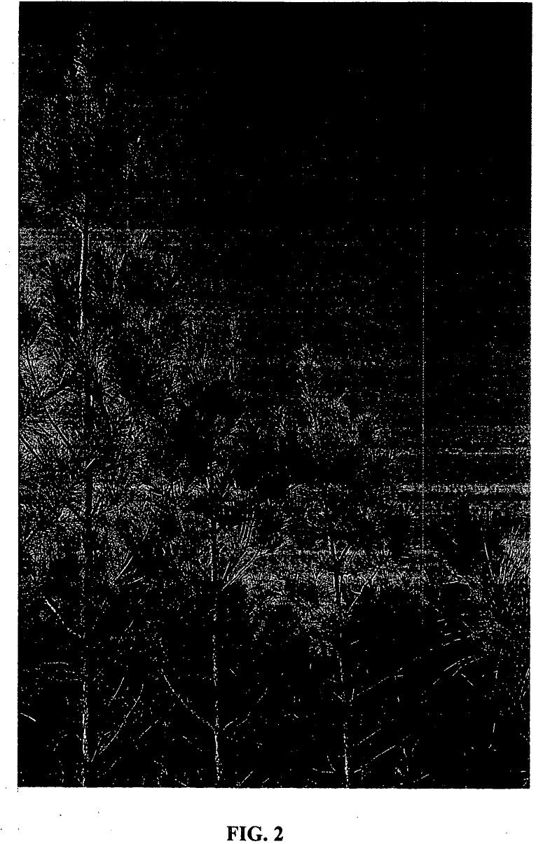

### DETAILED BOTANICAL DESCRIPTION

The botanical details of this new and distinct variety of loblolly pine tree follow.
Leaf: Evergreen needles, 6 to 9 inches long, with (usually) three yellow-green needles per fascicle.
Flower: Monoecious; males long cylindrical, red to yellow, in clusters at branch tips; females yellow to purple.
Fruit: Ovoid to cylindrical, 3 to 6 inch red-brown cones; umbo is armed with a short spine, maturing in early fall.
Twig: Orange-brown in color, fine to moderately stout; buds are narrowly ovoid, light reddish brown.
Bark: Initially red- to gray-brown and scaly; older trees are ridged and furrowed, with somewhat rounded scaly plates; very old trees have red-brown, flat scaly plates.
Form: A medium to large tree can reach well over 100 feet tall, self-prunes well and develops a fairly straight trunk and an oval, somewhat open crown. Compared to unimproved loblolly pine trees, ‘CF Q7766’ is characterized by exceptionally high growth rate, great resistance to fusiform rust (caused by Cronartium quercuum (Berk.) Miyabe ex Shirai f. sp. fusiforme (Cumm.) Burds. et Snow), great resistance to pitch canker (caused by Fusarium circinatum Nirenburg et O'Donnell), distinctive very long internodes, excellent stem straightness, narrow crown, small to medium branch diameter, flat to medium branch angle and low incidence of forking.
Average height: 28 ft after 5 field growing seasons
Maximum height: 34 ft after 5 field growing seasons
Average diameter at breast height: 4.7 inches after 5 field growing seasons
Maximum diameter at breast height: 6.4 inches after 5 field growing seasons
Percent stem forking at age 4: 2
Percent ramets with ramicorn branches at age 4: 2
Percent stem fusiform rust infection at age 5: 0
Percent branch fusiform rust infection at age 5: 5
Percent branch and stem fusiform rust infection at age 5: 0
Percent dead ramets due to fusiform rust infection at age 5: 0 Percent stem fusiform rust infection in the USDA Resistance Screening Center (Asheville, N.C.) tests after artificial inoculation with rust spores: 5% (compared to 78% infection in unimproved seedlings) Percent of seedlings with over 50% of tissue infected by pitch canker in the USDA Resistance Screening Center (Asheville, N.C.) tests after artificial inoculation with pitch canker spores: 1% (compared to 99% infection in unimproved seedlings)
Percent stem straightness gain over unimproved trees: 15
Branch angle: Flat to medium
Branch diameter: Small to medium
Number of major whorls: Few
Live crown width: Narrow
Propagation: Propagated by somatic embryogenesis
Use: High yield industrial plantations

Although the new variety of loblolly pine tree possesses the detailed characteristics noted above as a result of the growing conditions prevailing in the seven test locations, it is to be understood that the variations of the usual magnitude and characteristics incident to changes in growing conditions, irrigation, fertilization, pruning, pest control, climatic variations and the like are to be expected. An example of ‘CF Q7766’ can be found at Plum Creek Oliver year 2001 line trial, Screven county, GA.

Having thus described and illustrated our new variety of loblolly pine tree, what we claim as new and desire to secure by Plant Letter Patent is:

1. A new and distinct variety of loblolly pine tree named CF Q7766 substantially as described and illustrated.
